(19) United States
(12) Patent Application Publication
(10) Pub. No.: US 2003/0132117 A1
(43) Pub. Date: Jul. 17, 2003

(54) Thin film electrophoresis apparatus and method

(76) Inventors: Andras Guttman, San Diego, CA (US); Bart Wanders, Carlsbad, CA (US); Phil Alei, Carlsbad, CA (US)

Correspondence Address: HALE AND DORR, LLP, 60 STATE STREET, BOSTON, MA, 02109

(21) Appl. No.: 10/047,461

(22) Filed: Jan. 14, 2002

(51) Int. Cl. G01N 27/26; G01N 27/447; G01N 33/48; G01N 33/50; G06F 19/00

(52) U.S. Cl. 204/620; 204/621; 204/616; 204/612; 204/470; 204/466; 204/461; 382/128; 382/129; 702/19; 702/20; 702/22; 702/32

(57) ABSTRACT

High throughput electrophoresis having a plurality of samples migrating through a highly resistive separatory media under an electric field. Multiple electrophoresis runs are conducted using a single separatory media. Automated methods of data analysis yields quantitative and qualitative data using optical densitometer or other detection systems. Controlled operating temperatures and small geometry reduces dispersion inaccuracy in banded data.

### RELATED APPLICATIONS

This application claims priority to U.S. Provisional Application serial No. xx/yyy,yyy arising from conversion of non-provisional U.S. patent application Ser. No. 09/759,989, (entitled NANOPOROUS MEMBRANE REACTOR FOR MINIATURIZED REACTIONS AND ENHANCED REACTION KINETICS, and filed on Jan. 12, 2001), to a provisional application by petition filed in the U.S. Patent and Trademark Office on Jan. 4, 2002, the specification of which is herein incorporated by reference.

### TECHNICAL FIELD

This document relates generally to electrophoresis systems, devices and methods and particularly, but not by way of limitation, to an electrophoresis apparatus having high capacity and yielding high resolution data.

### BACKGROUND

Electrophoresis is an analytical tool for separating constituent elements of a sample. In particular, electrophoresis has been demonstrated to be effective for analysis of complex mixtures of molecules, such as proteins, peptides, amino acids, nucleic acids, inorganic ions, organic bases, organic acids, whole cells, deoxyribonucleic acid (DNA), and others. Electrophoresis can provide data concerning the mobility, size and charge of a molecule.

In brief, electrophoresis entails moving an unknown sample through a porous medium. The various constituent elements migrate through the separation media at different rates depending upon their molecular properties, electrical charge to mass ratio and other factors. If the separation media is a gel, then one such factor is the size of the molecule. An external electric field, such as that provided by a DC power supply, may be applied to the separation media to promote migration of the sample.

In one form of electrophoresis, the constituent elements appear as bands within the porous media, or separatory media. Typically, the separatory media, or gel, includes agarose or other gelatinous compounds. At high temperatures, agarose is a fluid and may be cast into a mold to create a porous media suitable for electrophoresis when cooled to room temperature.

Various systems and techniques have been developed for detecting the bands created by the constituent elements within the separatory media. Examples of detection systems include measuring the electrical conductivity (or resistance) of each band, or measuring fluorescence or optical density based on light absorption of each band. An optical densitometer detection system includes a light source, often a laser, and optical detectors to measure emitted light. The quantitative output of the band detection system yields the identity of the constituent elements, and thus, the composition of the sample. Detectors may include electrochemical or radiochemical systems.

Traditional electrophoresis is not renowned for high resolution or rapidity. Resolution is a measure of the quality of the separation of the bands and, in general, broad bands yields low resolution. The speed with which results can be achieved is based, in part, on the migration rate of the constituent elements. The field strength of an external electric field is limited by the physical dimensions of the separatory medium. If the electric field strength is too high, then the sample may be destroyed by excessive heat.

Therefore, there is a need for an improved electrophoresis system and method that yields high resolution and rapid results.

### SUMMARY

The present subject matter is directed to apparatuses, systems and methods for performing analysis by electrophoresis. In one embodiment, the apparatus includes two fluid reservoirs coupled by a narrow chamber. The chamber includes parallel top and bottom plates and is filled with a separatory media. A void formed in one end of the medium located near one of the reservoirs is adapted to receive a comb-like membrane having approximately 100 teeth. One or more of the teeth of the membrane is spotted with a sample to be analyzed.

In operation, the reservoirs contain a running buffer solution and an electric field is generated within the separatory medium by electrodes coupled to each of the reservoirs. An optical densitometer, or other band detector, provides graphical data concerning migration of the constituent elements of the samples. The graphical data includes one or more bands arranged in each of a plurality of discrete lanes where each lane is associated with a tooth of the membrane.

In one embodiment, the apparatus is fabricated of glass, plastic, or other transparent material and the plates of the chamber are spaced apart by approximately 190 microns.

The present subject matter also concerns a method of analyzing banded data arranged in a three dimensional grid system, in one embodiment. The three dimensions include, for example, an x-axis (spatial), a y-axis (time) and a z-axis (signal intensity). In a first step, each of a plurality of lanes are detected using a peak detection algorithm. The boundaries of each lane may be determined based on the dimensions of the membrane or by other means. In a second step, within each detected lane, each of a plurality of bands are detected using a peak detection algorithm. The peak detection algorithm may operate on data from the optical densitometer or other detector.

In one embodiment, the separatory medium includes a gel formed within the chamber by heating and casting in situ. In one embodiment, the molten gel is poured into a reservoir and forced into the chamber by means of air pressure. A temporary spacer element may be inserted into the chamber to form a void to receive the membrane.

In one embodiment, automated methods are used for spotting the teeth of the membrane, inserting the membrane and applying the fluids to perform electrophoresis.

Other aspects of the invention will be apparent on reading the following detailed description of the invention and viewing the drawings that form a part thereof.

### BRIEF DESCRIPTION OF THE DRAWINGS

In the drawings, like numerals describe substantially similar components throughout the several views. Like numerals having different letter suffixes represent different instances of substantially similar components.

FIG. 1 is a sectional view of one embodiment of an electrophoresis apparatus.

FIG. 2 is a perspective view of one embodiment of an electrophoresis apparatus.

FIGS. 3A and 3B illustrate one embodiment of a membrane.

FIGS. 4A and 4B are sectional views of a portion of a membrane.

FIG. 5 is a view of a portion of an electrophoresis apparatus.

FIG. 6 is a sectional view of a portion of one embodiment of an electrophoresis apparatus.

FIG. 7 is a sectional view of a portion of one embodiment of an electrophoresis apparatus.

FIG. 8 is a view of a spacer in accordance with one embodiment of the present subject matter.

FIG. 9 is a perspective view of a tool for use with one embodiment of an electrophoresis apparatus.

FIG. 10 is a perspective view of a tool coupled to a partially assembled electrophoresis apparatus.

FIG. 11 is a perspective view of an electrode assembly for use with one embodiment of an electrophoresis apparatus.

FIG. 12 illustrates a flow chart of a method for preparing and operating an electrophoresis apparatus.

FIG. 13A is a photograph of a screen displaying data derived from an electrophoresis apparatus according to the present subject matter.

FIG. 13B illustrates selected details of the data shown in FIG. 13A.

FIG. 14 illustrates a flow chart of a method for analyzing banded data from an electrophoresis apparatus.

FIG. 15 illustrates a flow chart of a method for analyzing banded data from an electrophoresis apparatus.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description is, therefore, not to be taken in a limiting sense, and the scope of the present invention is defined by the appended claims and their equivalents.

By way of overview, the present system includes a pair of reservoirs coupled by a narrow chamber. Within the narrow chamber is a separatory media, or a gel in one embodiment. Samples to be analyzed are spotted onto the teeth of a comb-like membrane having approximately 100 teeth. The membrane is inserted into a void formed in the separatory medium and running buffer solutions are introduced into the reservoirs. The running buffer solutions provide pH buffering and conducts an electric field through the separatory medium.

The constituent elements of each sample migrate away from the membrane and through the separatory medium based on various factors including the resistance through the medium, the electric field strength and the size, electric charge and configuration of each constituent element. Data concerning the migration of the constituent elements can be determined by optical densitometer or other band detection methods.

In one embodiment, the resulting bands are analyzed using a method which yields quantitative data. In one embodiment, the resulting bands are analyzed using a method which yields qualitative data. Each tooth of the membrane bearing a sample forms a discrete lane within the separatory medium. Each lane may have one or more bands corresponding to each of the constituent elements of the sample on that particular tooth of the membrane. One method entails, first, detecting the lanes, and second, detecting bands within each lane. In one embodiment, lanes are detected by summing the data points along a first axis, normalizing the data and determining the peaks using a peak detection algorithm. Within each detected lane, the process is repeated to detect bands. In particular, band detection includes summing the data points along a second axis and determining the peaks using a peak detection algorithm.

The membrane described herein is an example of a sample delivery device. Other sample delivery devices are also contemplated, including manual devices for providing a sample to the separatory medium of the present subject matter.

The present subject matter is adapted for large volume analysis with high resolution output. It is believed that the narrow chamber dimensions provides a uniform environment for migration of the constituent elements of the samples. The chamber also presents a high resistance media for the constituent elements and thus, reduces temperature gradations. The present subject matter allows application of a high intensity electric field thus yielding high resolution data with rapid migration.

Structure

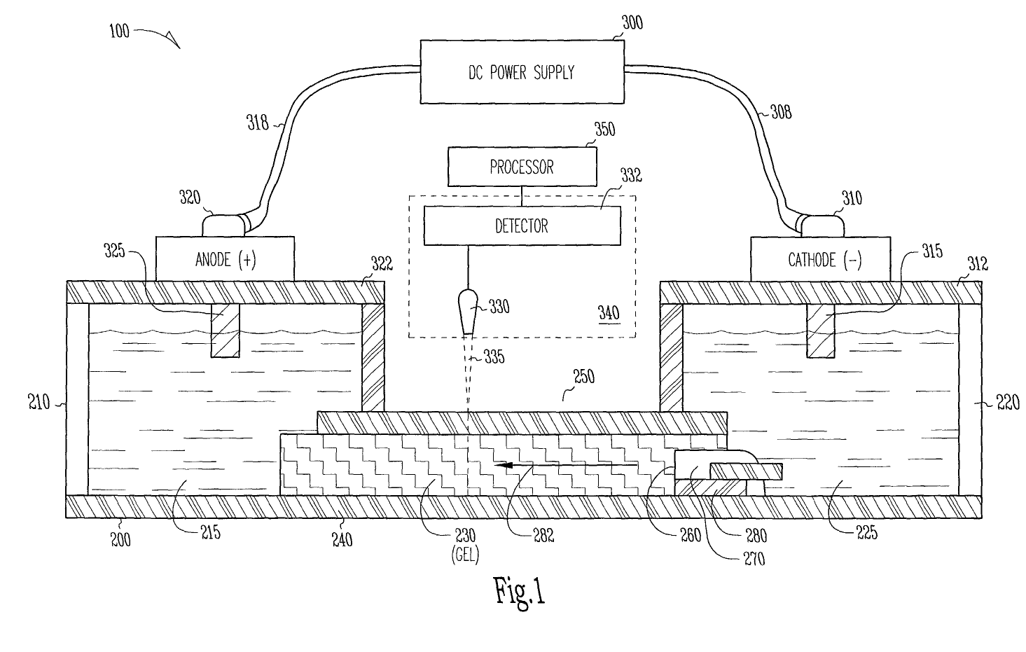

FIG. 1 illustrates a sectional view of one embodiment of system 100 for conducting electrophoresis analysis in accordance with one embodiment of the present subject matter. Not shown in the figure are end caps for the reservoirs and the chamber and other structure.

In one embodiment, device 200 includes reservoir 210 and reservoir 220. Reservoirs 210 and 220 are coupled by a chamber which is bounded by upper plate 250 and lower plate 240. The chamber contains gel, or other separatory media, 230 and at least a portion of membrane 280. Reservoir 210 is capped with cover 322 and reservoir 220 is capped with cover 312. In one embodiment, cover 322 is coupled to anode 325 and cover 312 is coupled to cathode 315. In one embodiment, cover 322 is coupled to cathode 315 and cover 312 is coupled to anode 325. Anode 325 and cathode 315 are coupled to DC power supply 300 by leads 318 and 308, respectively. An electrical connection between anode 325 and lead 318 is established by connector 320. An electrical connection between cathode 315 and lead 308 is established by connector 310. It will be noted that anode 325 and cathode 315 may be positioned in a manner separately from covers 322 and 312, respectively. For example, anode 325 and cathode 315 may include flat conductors positioned within the respective reservoirs and bonded to lower plate 240.

In one embodiment, detection system 340 includes an optical detector 330 coupled to an optical densitometer 332. Densitometer 332 is coupled to processor 350. Other embodiments are also contemplated, such as for example, a fluorescence detection system. Processor 350, in one embodiment, provides data analysis as described elsewhere in this document.

Device 200 may be fabricated of translucent or opaque material including plastic, glass or other materials. In one embodiment, either upper plate 250 or lower plate 240 or both are fabricated of material compatible with detection system 340. In one embodiment, reservoir 210, reservoir 230, upper plate 250 and lower plate 240 are separately fabricated and bonded together in the manner illustrated to form leakproof joints.

In one embodiment, cover 322 and cover 312 protect the contents of reservoirs 210 and 220 from evaporation, foreign objects, and other contaminants. Covers 322 and 312 need not fit reservoirs 210 and 220, respectively, with a fluid tight seal.

In one embodiment, cover 322 and cover 312 carry electrical terminals. For example, anode 325 is coupled to cover 322 and cathode 315 is coupled to cover 312. With cover 322 in position on reservoir 210, anode 325 is in electrical contact with running buffer 215 and with cover 312 in position on reservoir 220, cathode 315 is in electrical contact with running buffer 225.

Separatory medium 230 occupies the chamber between upper plate 250 and lower plate 240. In one embodiment, upper plate 250 and lower plate 240 are substantially parallel and spaced apart by a distance of 190 microns. Distances greater than or less than 190 microns are also contemplated. In one embodiment, separatory medium 230 is formed in place by heating to a melting point and forcing into the space between upper plate 250 and lower plate 240.

Separatory medium 230 may include agarose or a gel known to one of skill in the art. In one embodiment, the separatory medium is pure agarose (in the range of, for example, 0.05-5% or 0.01 to 30%). In one embodiment, the separatory medium includes a composite. An example of a composite includes agarose and linear polyacrylamide (LPA) gel (with agarose in the range of, for example, 0.05-5%, or 0.01 to 30% and LPA in the range of, for example, 0.05-10%). In one embodiment, the separatory media is adapted to separate molecules in various molecular weight ranges. In various embodiments, the weight range is between 1,000 to 10,000,000 or 10,000 to 1,000,000,000 although other weight ranges are also contemplated.

Resolution of electrophoresis data is believed to be affected by temperature gradations across the separatory medium and the strength of the electric field through the medium. Here, the narrow dimensions of the chamber yields lower temperature variability, and thus, sharper bands of data with higher resolution. Higher temperatures cause the samples to move more rapidly, leading to more regularly shaped bands and less dispersion or blurred images. Thus, with less temperature gradients, the resolution improves since the bands remain sharp and narrow.

In addition, it is believed that the narrow dimensions of the chamber permit the use of a greater external electric field strength. For example, in one embodiment, the external voltage provided by supply 300 may be in the range of 50 to 100 volts per cm, with a typical value of 75 volts per cm. Thus, over a 10 cm distance, the supply voltage may be 1,000 volts. External cooling of device 200 may allow voltages greater than 100 volts per cm. For example, liquid cooling provided to upper plate 250 or lower plate 240 may allow use of a greater external field, and thus, higher resistance pathways and therefore, yield higher throughput. Liquid cooling may include water cooling or circulating a cooling agent or fluid in the proximity of the separatory medium. When using approximately 75 volts per cm to provide an external field, the run time for system 100 is typically 5 to 25 minutes in duration.

Electro osmotic forces (EOF) arising from application of the external electromagnetic field, may tend to move separatory medium 230 from within the chamber. In one embodiment, separatory medium 230 includes an additive that dynamically coats the interior surface of the chamber, thereby attenuating such electro osmotic forces. One such additive includes a polymer, such as polyvinylpiperidone (PVP), or dimethylacrylamide or other hydrophilic linear polymer.

In the absence of a coating on the interior of the chamber, the electrostatic charge of the fluid is generally positive fluid and the charge of the surface of the chamber is generally negative. Using a polymer coating yields a neutral surface and a neutral fluid, thus reducing electro osmotic forces.

Void 260 is formed in one end of separatory medium 230 located near reservoir 220 and at least partially within the space between upper plate 250 and lower plate 240. Void 260 is adapted to accept membrane 280 and a quantity of focusing water 270.

The constituent elements of the samples delivered to separatory medium 230 by membrane 280 travel in the direction indicated by arrow 282. The constituent elements are repelled by the cathode 315 end and attracted to anode 325 end.

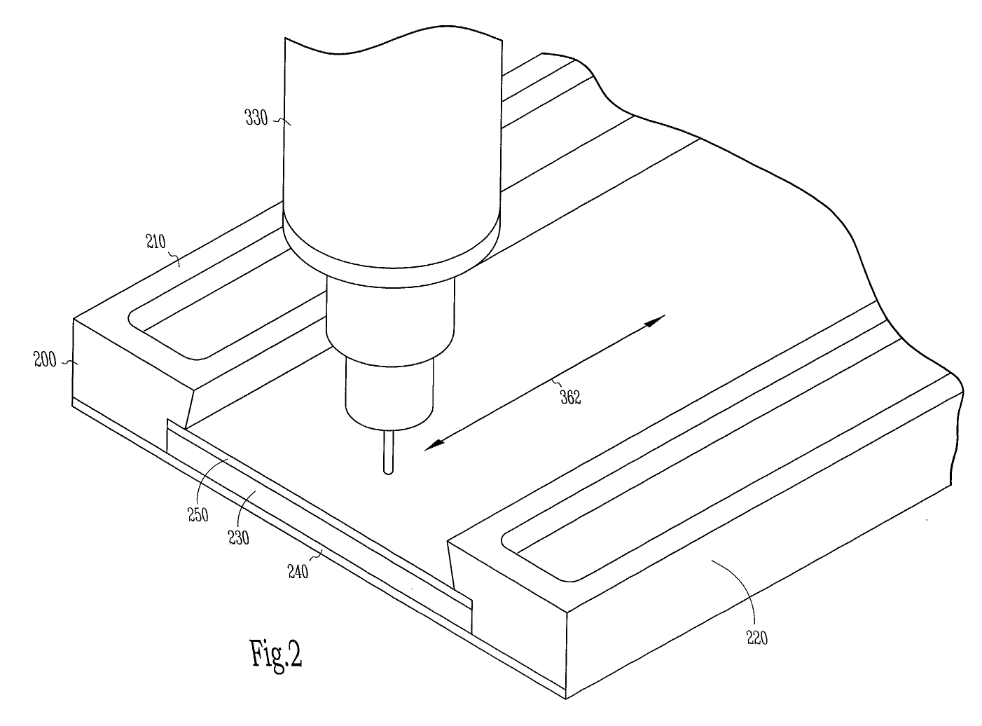

FIG. 2 illustrates a perspective view of one embodiment of device 200 with cover 312 and cover 322 removed for the sake of clarity. In the embodiment illustrated, reservoir 210 and reservoir 220 are machined from a solid plastic block. Reservoir 210 and reservoir 220 are coupled to upper plate 250 and lower plate 240.

In the figure, optical densitometer detector 330 is positioned at a near end of separatory medium 230. Detector 330 is mounted on a track mechanism (not shown) and travels in the directions indicated by the arrowheads on line segment 362. In one embodiment, detector 330 includes a central optical fiber for transmitting a column of light and a plurality of sensor fibers distributed about the circumference of the central optical fiber for receiving reflected light. In cycling back and forth, detector 330 provides a signal based on the intensity of the bands generated by the constituent elements as each migrates through separatory medium 230. The output of detector 330 is processed by detector system 332 of FIG. 1 and further processed by processor 350, also of FIG. 1. In one embodiment, device 200 is mounted to a commercial stage apparatus and detector 330 is powered by a motorized carriage mechanism coupled to the stage.

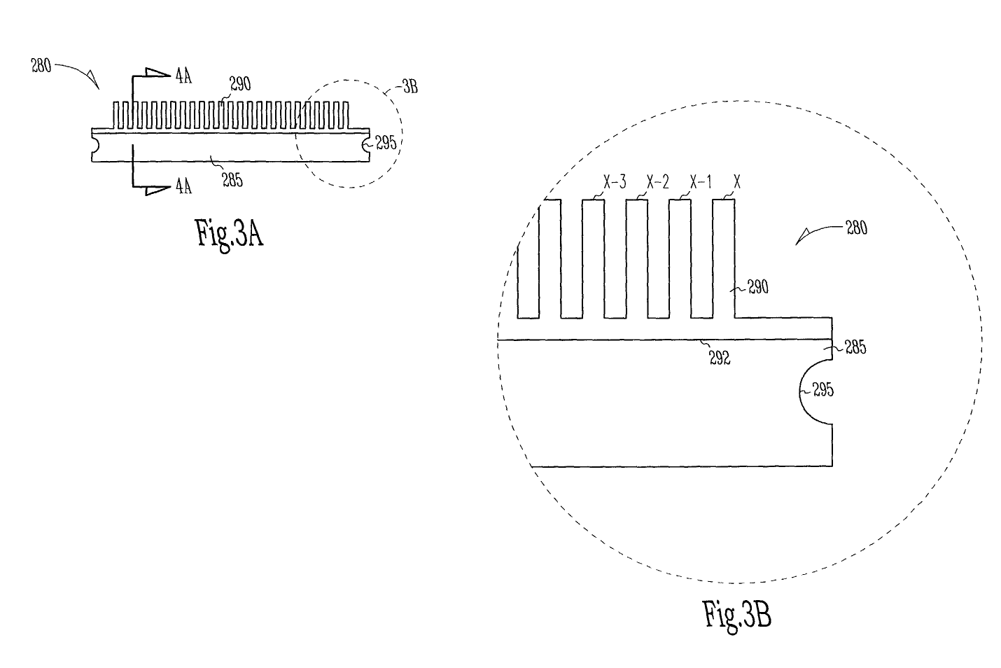

FIGS. 3A and 3B illustrate views of membrane 280. In FIG. 3A, membrane 280 includes comb 290 and reinforcer 285. Reinforcer 285 includes alignment notches 295 at each end.

FIG. 3B illustrates a close view of membrane 280. Comb 290 includes a plurality of teeth, some of which are visible in the view shown and are denoted herein as X, X−1, X−2, X−3. In various embodiments, X is 96 or 100. In one embodiment X is an integer between 50 and 150. Comb 290 is bonded to reinforcer 285, forming a laminate structure having visible joint line 292. Alignment notch 295 is visible in FIG. 3B.

In one embodiment of system 100, membrane 280 is installed in device 200 in such a manner that the thicker portion of comb 290 and reinforcer 285 is positioned under reservoir 220 and a portion of the teeth of comb 290 are inserted within the space between upper plate 250 and lower plate 240. Other embodiments are also contemplated, such as for example, insertion of membrane 280 to a greater or lesser depth.

In one embodiment, membrane 280 includes a porous or nanoporous structure. Methods, materials, devices and systems concerning nanoporous membranes are described in U.S. patent application Ser. No. aa/bbb,bbb, entitled NANOPOROUS MEMBRANE REACTOR FOR MINIATURIZED REACTIONS AND ENHANCED REACTION KINETICS, filed Jan. 14, 2002, invented by Andras Guttman, Zsolt Ronai, and Csaba Barta and assigned to Syngenta Participation AG, the specification of which is incorporated herein by reference.

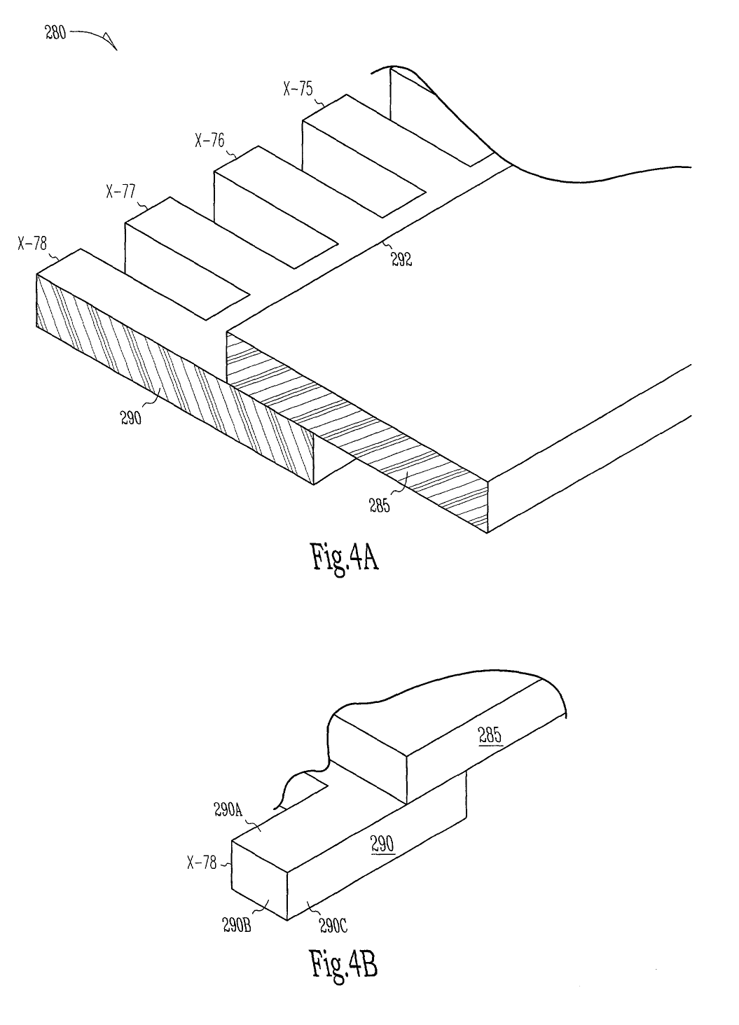

FIG. 4A illustrates a perspective view of a portion of membrane 280 when viewed in the direction of cut-line A-A shown in FIG. 3A. The bond line between comb 290 and reinforcer 285 and joint line 292 are visible in the figure. In FIG. 4A, teeth X−78, X−77, X−76 and X−75 of comb 290 are shown as rectangular teeth, however, it will be appreciated that other shapes and configurations are also contemplated. For example, the teeth of comb 290 may have a circular cross-section or include rounded ends in contrast to the square cuts shown. It will be appreciated that comb 290 is illustrated as having teeth of uniform dimensions and features, however, alternative configurations are also contemplated. For example, the teeth may have progressively shorter dimensions or have larger area or include triangular, tapered, or pyramid shaped teeth.

FIG. 4B illustrates a perspective view of representative tooth X−78 of comb 290 coupled to a portion of reinforcer 285. The sample to be analyzed by electrophoresis may be deposited on one or more of surfaces 290A, 290B and 290C. In one embodiment, the sample is deposited onto membrane 280 robotically. On any particular comb 290, each tooth may carry a unique sample or some teeth may carry the same sample.

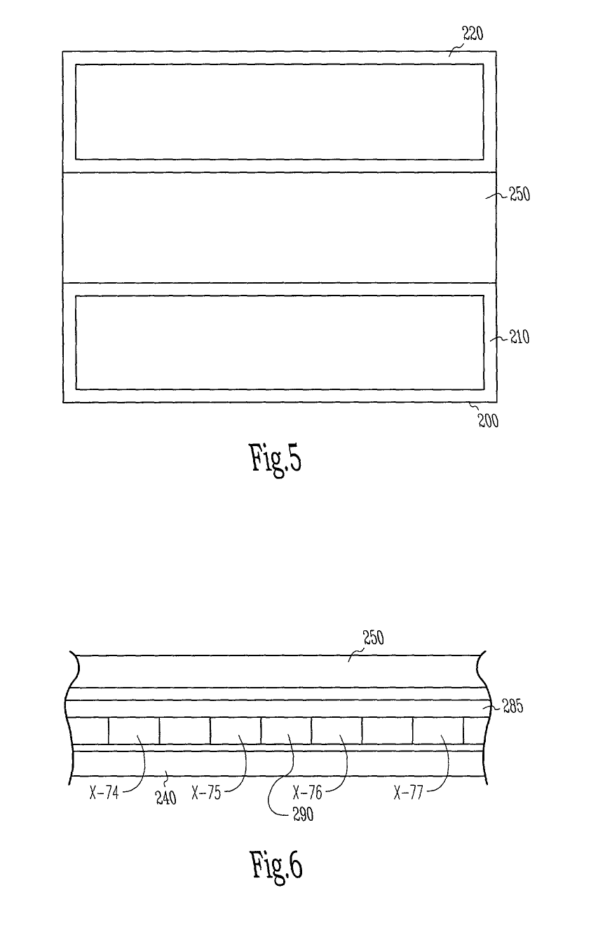

FIG. 5 illustrates a top view of device 200 with cover 322 and cover 312 removed and before installation of separatory medium 230 and membrane 280. Visible in the figure are the boundary walls of reservoir 210 and reservoir 220 coupled by top plate 250.

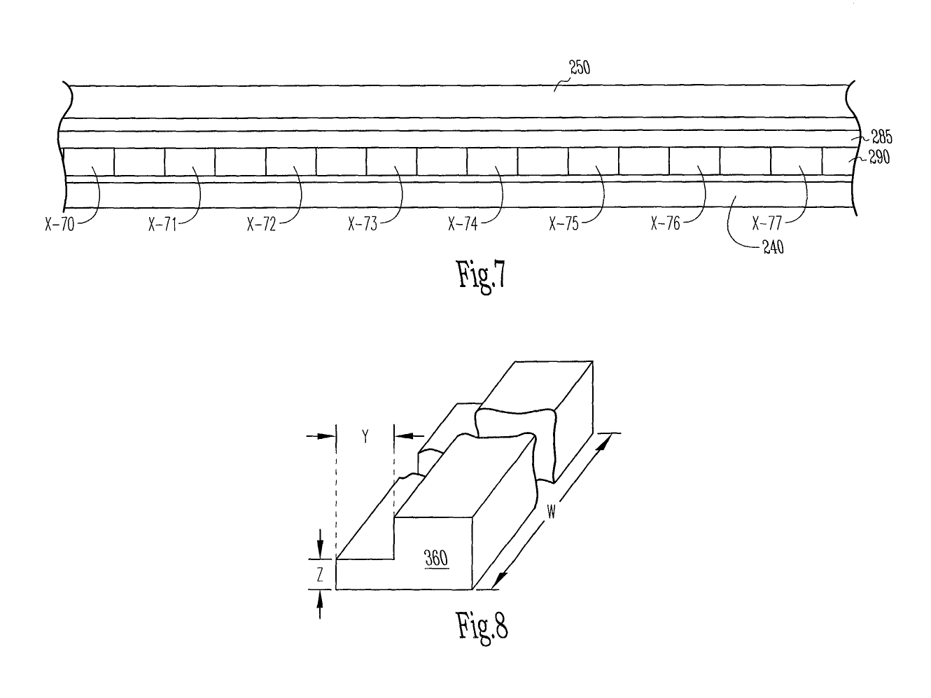

FIG. 6 illustrates a view of a portion of membrane 280 having comb 290 and reinforcer 285. Upper plate 250 and lower plate 240 are shown to be of substantially the same thickness dimension and are spaced apart, in one embodiment, at a distance of 190 microns. In one embodiment, upper plate 250 and lower plate 240 may be of different thickness dimensions with one greater than the other. In one embodiment, the combined thickness of laminated structure 280, including comb 290 and reinforcer 285, is approximately 130 microns and the length of the portion of comb 290 inserted into the chamber between upper plate 250 and lower plate 240 is approximately 1 to 2 mm.

To improve the accuracy of data generated by device 200, it is preferable that upper plate 250 and lower plate 240 are substantially parallel. If the space between upper plate 250 and lower plate 240 is too small, then insertion of membrane 280 may be precluded. In addition, the resistance of the separatory medium 230 will vary with location and thus, the current flow through separatory medium 230 may be irregular. Irregular current flow leads to different speeds of constituent element migration, thus impairing the value of the data generated.

Maintaining uniform and accurate separation of the plates 250 and 240 over the width of the chamber (which, in one embodiment is a distance of 12 to 25 cm) may be accomplished using any one of, or a combination of, techniques. For example, at the time of manufacturing of device 200, a suitable temporary spacer may be inserted between the lower plate 240 and upper plate 250 before bonding of reservoirs 210 and 220 to plates 240 and 250. After the bonds have cured, the temporary spacer is removed and device 200 is ready for installation of the separatory medium. In one embodiment illustrated in FIG. 7, the thickness dimension of upper plate 250 is increased relative to that of lower plate 240.

The dimensional stability of the upper plate 250 relative to lower plate 240 is derived from external means. For example, stability may be provided by the structural strength of reservoirs 210 and 220 or by the bond between reservoirs 210 and 220 and the upper plate 250 and lower plate 240.

Method

As noted above, separatory medium 230 preferably presents a uniform resistance to migration of the constituent elements of the sample. The following discussion concerns a method for installing the separatory medium into the chamber between upper plate 250 and lower plate 240.

In brief, one embodiment provides that the separatory medium is heated to more easily flow into the narrow chamber, poured into either reservoir 210 or reservoir 220 and pumped through the chamber under pneumatic pressure. Void 260 is prepared by inserting a suitable spacer into separatory medium 230 while still molten.

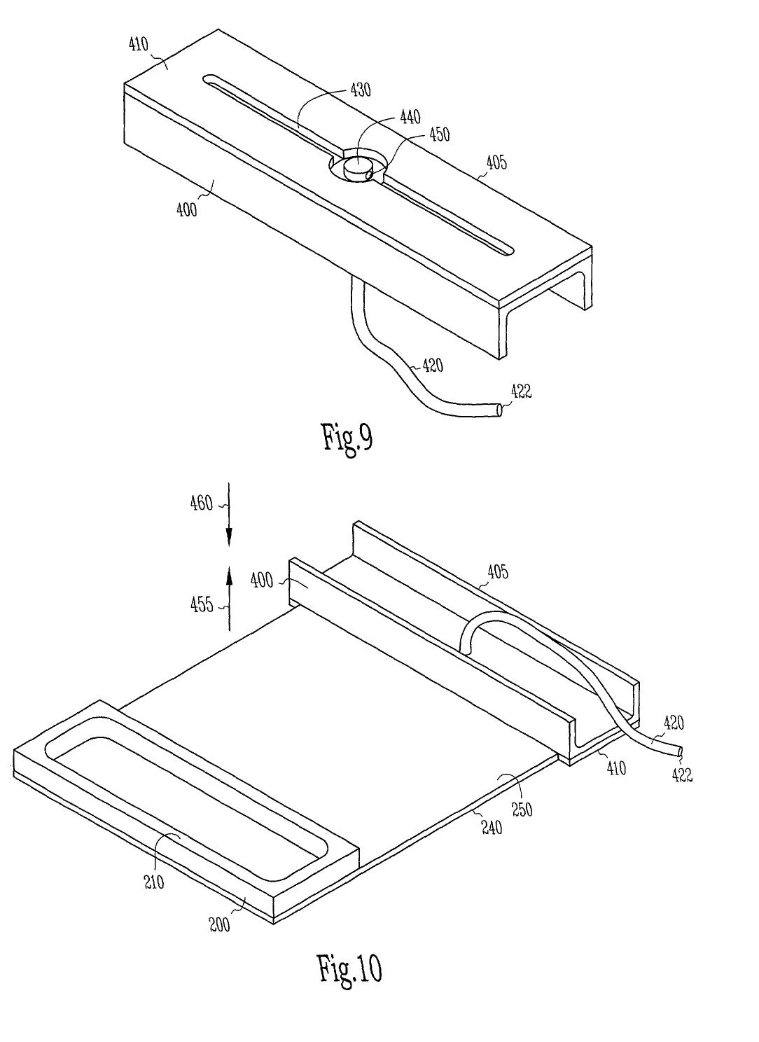

FIG. 8 illustrates a portion of spacer 360 that may be used for preparing void 260 in separatory medium 230. In one embodiment, dimension Z is approximately 150 microns and dimension Y is approximately 3 to 5 mm and width dimension W is approximately 15 cm, however, other dimensions are also contemplated. Dimensions Z, Y and W are selected to provide a suitable space for insertion of membrane 280 into separatory medium 230. Spacer 360 may be fabricated of plastic, metal or other suitable material. In one embodiment, spacer 360 is made of plastic shim stock and includes a thicker portion to allow easy manipulation for insertion and removal.

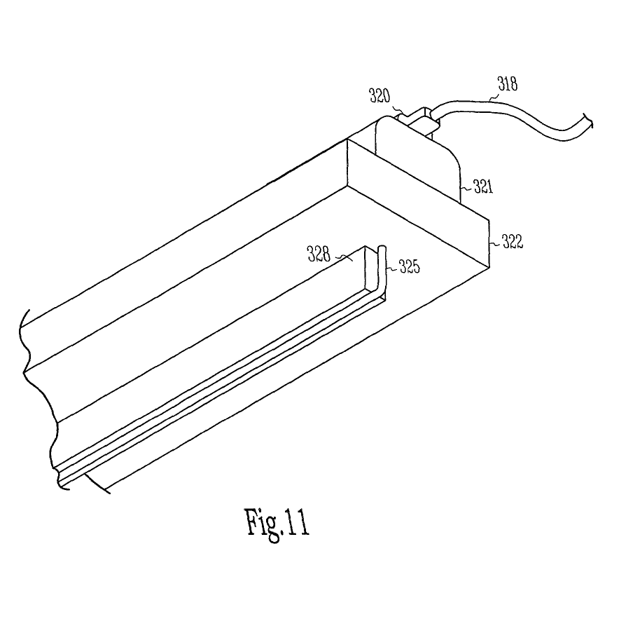

FIG. 9 illustrates apparatus 405 that may be used to apply pneumatic pressure to a molten separatory medium 230. In one embodiment, apparatus 405 includes gasket 410 bonded to rigid channel 400. Channel 400 may include an extruded or formed aluminum or plastic structure. Gasket 410 may include a rubberized surface of approximately 0.5 cm thickness. Tube 420 is coupled at a first end to fitting 440 and at a second end 422 to a source of pressurized air. Fitting 440 may be threadably engaged with channel 400 and includes port 450 which communicates pressurized air to manifold 430 along the length of channel 400. Apparatus 405 may be fabricated using other structures and materials.

In one embodiment, separatory medium 230 may be moved into position by application of a vacuum source. Apparatus 405 may be coupled to a vacuum source and coupled to device 200 in a manner that draws separatory medium 230 into the chamber.

FIG. 10 illustrates apparatus 405 mounted on device 200. Apparatus 405 is positioned with gasket 410 in contact with reservoir 220. Gasket 410 is compressed between channel 400 and reservoir 220 by a clamping force. In one embodiment, apparatus 405 may be urged in the direction of arrow 460 by a pair of fixture clamps attached to a surface on which device 200 is placed. Thus, device 200 and apparatus 405 are urged in the directions shown by arrows 455 and 460, respectively.

Air pressure introduced into flexible tube 420 is distributed through port 450 and manifold 430 to reservoir 220. Molten separatory medium 230 in reservoir 220 is pneumatically forced into the space between upper plate 250 and lower plate 240.

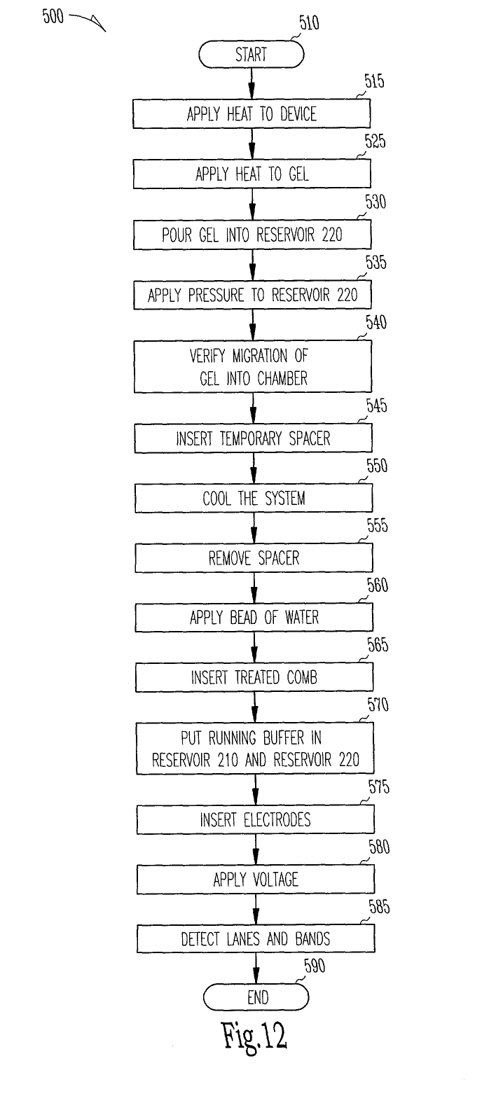

FIG. 11 illustrates cover 322. Cover 322 serves as a lid for reservoir 210 and carries anode 325. Cover 322 includes standoff 328 and electrical conductor 325, mounting block 321 and terminal 320 coupled to electrical lead 318. Cover 322 and standoff 328 may be fabricated of insulative material such as plastic, glass or other suitable material. Cover 322 and standoff 328 are bonded together. Terminal 320 provides an electrical connection between lead 318 and conductor 325. In the embodiment shown, conductor 325 is a bare wire laced through a hole in cover 322 and along the length of standoff 328.

Preparation

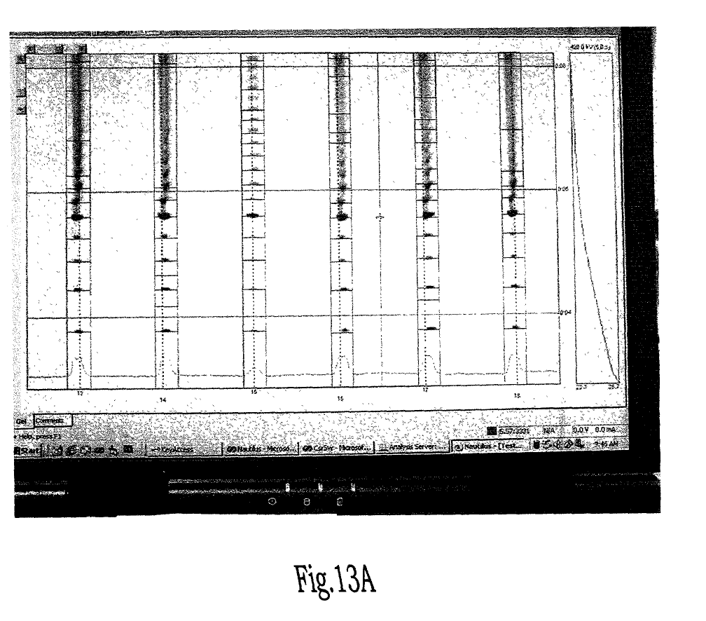

FIG. 12 illustrates flowchart 500 for preparing and conducting electrophoresis analysis using one embodiment of apparatus 200.

The method begins at 510, and at 515, device 200 is heated to a temperature of approximately 60° C. At 525, separatory medium 230 is heated to a molten state at a temperature of approximately 60° C. In one embodiment, separatory medium 230 is viscous fluid at approximately 60° C. and relatively solid at 32° C.

At 530, separatory medium 230 is poured into reservoir 220. At 535, pneumatic pressure is applied to reservoir 220 using apparatus 405 as illustrated in FIGS. 9 and 10. Pneumatic pressure may be provided by a regulated source of air pressure such as air compressor. At 540, migration of separatory medium 230 may be verified by visually observing a contiguous line of separatory medium 230 emerging from reservoir 210. After verifying that separatory medium 230 has migrated through the space between upper plate 250 and lower plate 240, apparatus 405 may be removed from device 200. In one embodiment, device 200 is positioned with a slight incline from horizontal, thus bringing gravity to bear on the migration of separatory medium 230.

At 545 and at a time while separatory medium 230 is at an elevated temperature, temporary spacer 360 may be inserted into separatory medium 230 accessible through reservoir 220. In one embodiment, spacer 360 penetrates into the separatory medium for a depth of approximately 3 to 5 mm.

At 550, system 100, including device 200 and separatory medium 230, is allowed to cool to room temperature. Cooling may take approximately five to ten minutes and may be accelerated by refrigeration. In one embodiment, system 100 is cooled to a temperature where separatory medium 230 is no longer molten.

At 555, spacer 360 is removed from separatory medium 230, thus forming void 260. In one embodiment, void 260 provides a depth stop for the insertion of membrane 280.

The foregoing procedure describes one example of a method for making a separatory medium suitable for use with system 100. Other procedures, or sequences of steps may be implemented.

Separatory medium 230 formed in the manner described above may be used for multiple electrophoresis runs. For example, in one embodiment, a single separatory medium 230 may be used for all runs that may be completed in a day or other period of time.

Consider next the procedure for analyzing samples on a membrane. At 560, a small quantity of focusing water is injected into void 260. Ordinary water, having a very high resistance, is used for focusing water. Focusing water promotes an intense localized electric field, thus the bands tend to focus more rapidly.

At 565, membrane 280, having been previously treated with a sample to be analyzed, is inserted into separatory medium 230. Insertion may be accomplished manually or by robotic means, and in one embodiment, includes passing membrane 280 through reservoir 220. Membrane 280 is inserted with the teeth of comb 290 positioned proximate separatory medium 230 and reinforcer 285 distal from separatory medium 230.

At 570, running buffer 215 is introduced into reservoir 2 10 and running buffer 225 is introduced into reservoir 220. Running buffer 215 and running buffer 225 may be of the same or different composition. Running buffers 215 and 225 cover the exposed portions of separatory medium 230 and have a level sufficient to establish electrical contact with anode 325 and cathode 315, respectively.

At 575, anode 325 and cathode 315 are positioned to contact running buffers 215 and 225, respectively. In system 100 illustrated in FIG. 1, this includes placing covers 322 and 312 in position atop reservoirs 210 and 220, respectively.

At 580, an electrical potential is applied across anode 325 and cathode 315 from direct current (DC) power supply 300. Supply 300 may include a regulated voltage supply or a battery.

At 585, lanes and bands are detected within separatory medium 230. The bands may be detected using apparatus as shown in FIG. 1 or FIG. 2. In various embodiments, detecting bands may entail using a UV/Vis detector, a fluorescence detector, a conductivity detector, an electrochemical detector, a mass spectrometer, a radioactive detector, a post column reaction detector, an optical densitometer, or other types of detectors.

The procedure ends at 590.

Data Analysis

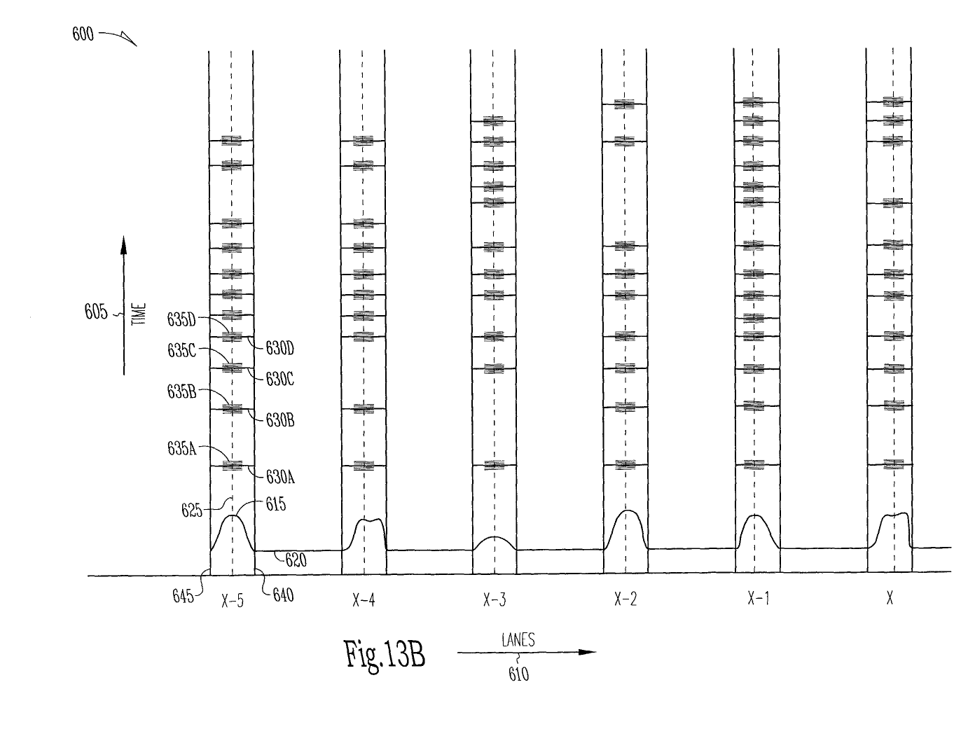

FIG. 13A depicts a photograph of a screen while displaying data derived from an electrophoresis apparatus according to the present subject matter. The photograph illustrates six lanes with each lane having a plurality of bands. In the photograph, the bands have migrated in a direction from bottom to top. In one embodiment, data is derived from more than six lanes, however, for purposes of clarity, only six lanes are depicted in the photograph.

FIG. 13B illustrates selected details of the data shown in FIG. 13A. The figure shows a screen shot of data derived from system 100 in one embodiment. Each lane, or column, of data appearing in the figure, herein denoted as X−5, X−4, X−3, X−2, X−1 and X, corresponds to a particular tooth, or lane, in comb 290. Since the present system is suitable for use with many samples, it will be appreciated that comb 290 may have many separate teeth, or lanes, and each lane corresponds to a sample arranged in the image in the form of a column. Thus, in one embodiment, a particular electrophoresis run may generate 100 lanes of data.

The bands appearing in the figure may be generated by an optical densitometer or other detection system.

In particular, consider column X−5. Within column X−5, eleven bands, or blots, appear. Each band denotes a particular constituent element of the sample carried by tooth X−5. With the possible number of lanes in excess of 100, analysis of even a single run may entail analysis of 1100 individual bands. It is believed that the present system may enhance reliability and accuracy of the analysis of such a large amounts of data.

The screen shot appearing in the figure indicates spatial data on the horizontal, or x-axis, denoted as axis 610, and time on the vertical, or y-axis, denoted as axis 605. A z-axis can be construed to represent the intensity of the signal. Data generated by the detection system 340 yields the individual bands. Representative bands appearing in the figure within column X−5 are denoted as 635A, 635B, 635C, 635C and 653D.

Detection system 340 is configured to acquire data at a rate sufficient to scan 100 lanes. For example, in one embodiment, the sampling rate of detection system 340 is approximately 2 kHz. In one embodiment, an external clock provides synchronization between data acquisition and movement of the scanning detector.

The picture in the figure represents a single line in the separatory medium (for example, at a position 10 cm from the injection side) plotted in time. Bands that migrate through the separatory medium fast will reach this line/detector, the earliest and are the first, or lowest, bands in the picture. Slower bands reach the detector later and thus, appear higher in the picture.

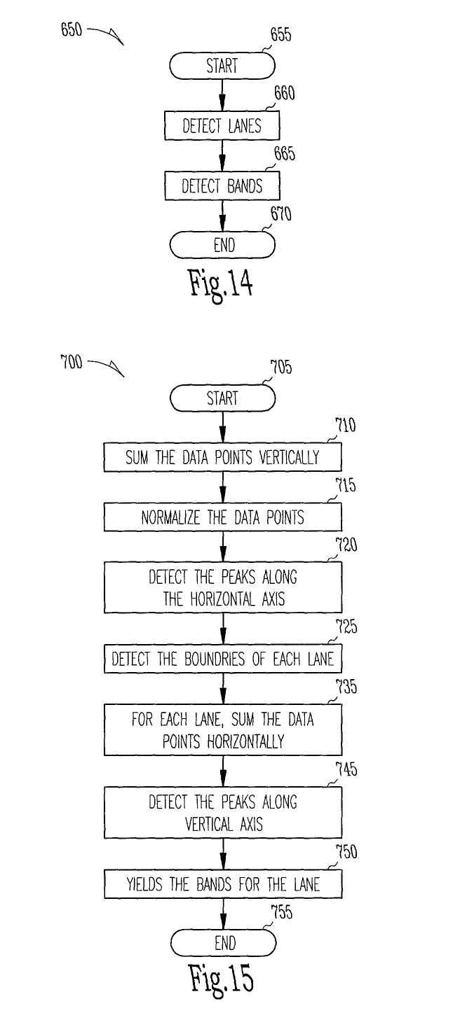

FIG. 14 illustrates one embodiment of method 650 for analyzing data. Beginning at 655, the method includes discerning the individual lanes from among a field of lanes, as at 660. At 665, and within each lane, the method also includes discerning individual bands. The method ends at 670.

FIG. 15 illustrates method 700 for analyzing data and begins at 705. To discern each individual lane, the method entails viewing the data as a two-dimensional picture. At 710, data points are summed along axis 610, that is between the bottom and the top of the image in FIG. 13B. For example, in column X−5, the data represented by each of the eleven bands are summed along the time axis. At 715, the data is normalized to reduce the range of data. At 720, and using the normalized data, a peak detection algorithm is used to identify individual lanes. In column X−5, for example, the peak occurs at point 615 and vertical dotted line 625 denotes the peak. At 725, and continuing with the example of column X−5, left boundary 645 and right boundary 640 on either side of the peak 615 (and line 625) are then constructed. In one embodiment, the position of boundaries 645 and 640 may be determined based on the physical dimensions of comb 290.

Having determined the boundaries for each lane, at 735, the data points are summed along axis 610. Then, data points are summed between left boundary 645 and right boundary 640. At 745, using a peak detection algorithm, each band is identified with a particular peak value. At 750, it is presumed that the peak ordinarily occurs at the most intense portion of each band, thus yielding the position for that particular band. However, the time value may be marked at either edge of the band or at another location relative to the band. In one embodiment, the peak, shown by vertical dotted line 625, is displaced to one side or the other. Further analysis can proceed using the center, peak, or any other location within the boundaries of the lane. In column X−5 of the figure, the eleven bands, some of which are denoted as 635A, 635B, 635C and 635D have values marked by lines 630A, 630B, 630C and 630D, respectively. The method ends at 755.

In one embodiment of the present subject matter, the methods described herein are implemented in executable computer software. For example, a personal computer coupled to a detector, such as an optical detector, is configured to execute programming to identify the lanes and to identify the bands within each lane. The software can be adapted to execute on a computer and stored on removable storage media.

Alternative Embodiments

In one embodiment, automation systems may be used to further increase throughput of samples. For example, an automatic membrane insertion tool may be used to robotically place membrane 280 in the chamber. Furthermore, robotic spotting equipment may place samples on the teeth of membrane 280. Robotic fluid dispensing equipment may be used to inject focusing water 270 or place running buffers 215 and 225 in their respective reservoirs. Timers or other robotic equipment may also be used to cycle power supply 300 to predetermined voltage levels. In one embodiment, an automated process may perform tens or hundreds of electrophoresis runs without human intervention.

Conclusion

The above-described system provides, among other things, a system, apparatus and method for performing electrophoresis with high resolution and high throughput while using a smaller quantity of chemicals.

It will be appreciated that the methods described herein may be performed in different orders than described and that portions of a method may be repeated.

It is to be understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

What is claimed is:

1. An apparatus comprising:
a first reservoir having a first buffer and a first electrode terminal;
a chamber coupled to the first reservoir and having a bottom plate and a top plate in parallel alignment and adapted to hold a separatory medium between the bottom plate and the top plate;
a second reservoir coupled to the chamber and having a second buffer and a second electrode terminal; and
a sample delivery device having a longitudinal axis and having a plurality of tabs extending orthogonally from the longitudinal axis, the sample delivery device adapted to couple with an opening of the second reservoir.

2. The apparatus of claim 1 wherein the top plate includes glass or plastic.

3. The apparatus of claim 1 wherein the bottom plate includes glass or plastic.

4. The apparatus of claim 1 wherein the top plate is separated from the bottom plate by less than 500 microns.

5. The apparatus of claim 1 wherein the top plate is separated from the bottom plate by approximately 190 microns.

6. The apparatus of claim 1 wherein the top plate has a first thickness and the bottom plate has a second thickness and wherein the first thickness is substantially greater than the second thickness.

7. The apparatus of claim 1 wherein the first electrode terminal is coupled to a first cover and wherein the second electrode terminal is coupled to a second cover.

8. The apparatus of claim 1 wherein the sample delivery device includes a membrane.

9. The apparatus of claim 1 wherein the sample delivery device includes a porous structure.

10. The apparatus of claim 1 wherein the sample delivery device includes a nanoporous membrane.

11. The apparatus of claim 1 wherein the plurality of tabs includes at least 5 tabs.

12. The apparatus of claim 1 wherein the plurality of tabs includes between 50 and 200 tabs.

13. The apparatus of claim 1 further comprising a band detector.

14. The apparatus of claim 1 wherein the band detector includes an optical densitometer.

15. The apparatus of claim 1 wherein the first electrode terminal is coupled to an anode or cathode of a power supply.

16. The apparatus of claim 1 wherein the second electrode terminal is coupled to an anode or cathode of a power supply.

17. A method of data analysis comprising:
receiving data points arranged in a coordinate system having an x-axis corresponding to one or more discrete samples, a y-axis corresponding to time and a z-axis corresponding to signal intensity of each data point;
detecting each of a plurality of lanes distributed along the x-axis, each lane associated with a particular sample of the one or more discrete samples; and
detecting one or more bands within each of the plurality of lanes.

18. The method of claim 17 further comprising summing the data points along the y-axis.

19. The method of claim 17 further comprising normalizing the data points along the x-axis.

20. The method of claim 17 wherein detecting each of the plurality of lanes includes detecting one or more peak values.

21. The method of claim 17 wherein detecting each of a plurality of lanes includes determining a first lateral boundary and a second lateral boundary for each lane.

22. The method of claim 17 wherein detecting one or more bands includes detecting one or more peak values.

23. The method of claim 17 wherein detecting one or more bands includes summing the data points along the x-axis within each of the plurality of lanes.

24. A method of analysis comprising:
along a first axis of a grid, summing a first plurality of data points;
along a second axis orthogonal to the first axis, detecting a first plurality of peaks among the sums of the first plurality of data points;
establishing a first boundary and a second boundary for each peak of the first plurality of peaks;
along the second axis, summing a second plurality of data points for each band located between a particular first boundary and a particular second boundary;
along the first axis, detecting a second plurality of peaks among the sums of the second plurality of data points between the particular first boundary and the particular second boundary; and
storing each peak of the second plurality of peaks.

25. The method of claim 24 further comprising along the second axis, normalizing the sums of the first plurality of data points,

26. The method of claim 24 further comprising receiving a plurality of data points distributed along the first axis and the second axis.

27. The method of claim 24 wherein establishing the first boundary and the second boundary includes receiving dimensional data for an electrophoresis comb.

28. A method comprising:
heating a separation medium to approximately a melting temperature;
placing the heated separation medium in a first reservoir of an electrophoresis device;
flowing the heated separation medium from the first reservoir into a chamber of the electrophoresis device;
inserting a spacer into the chamber from within a second reservoir of the electrophoresis device to create a void in the separation medium; and
cooling the separation medium.

29. The method of claim 28 wherein heating the separation medium includes heating to approximately 60° centigrade.

30. The method of claim 28 wherein flowing the heated separation medium from the first reservoir into the chamber includes migrating the heated separation medium into a space between an upper plate and a lower plate, the upper plate and lower plate connected to the first reservoir and the second reservoir.

31. The method of claim 28 wherein flowing the heated separation medium from the first reservoir into a chamber of the electrophoresis device includes pouring the heated separation medium into the first reservoir and pumping the heated separation medium into the chamber.

32. The method of claim 28 further including removing the spacer.

33. The method of claim 28 wherein flowing includes pumping the heated separation medium.

34. The method of claim 28 wherein flowing includes sealing the first reservoir and applying an elevated atmospheric pressure to the first reservoir.

35. The method of claim 28 further including verifying transfer of the separation medium through the chamber of the electrophoresis device into the second reservoir.

36. The method of claim 28 further comprising:
applying water to the void; and
inserting a membrane in the void.

37. A method comprising:
inserting a plurality of teeth of a toothed membrane into a void in a separatory media;
introducing running buffer into a first reservoir and a second reservoir; and
providing an electrical potential between the first reservoir and the second reservoir.

38. The method of claim 37 further comprising introducing water to the void before inserting the plurality of teeth.

39. The method of claim 37 further comprising circulating a cooling fluid near the separatory media.

40. The method of claim 37 further comprising applying one or more samples to one or more teeth of the plurality of teeth of the toothed membrane.

41. The method of claim 37 further including preparing a comb using a robotic spotter.

42. A computer readable storage media having instructions stored thereon for implementing a method comprising:
receiving data points arranged in a coordinate system having an x-axis corresponding to one or more discrete samples, a y-axis corresponding to time and a z-axis corresponding to signal intensity for each data point;
detecting each of a plurality of lanes distributed along the x-axis, each lane associated with a particular sample of the one or more discrete samples; and
detecting one or more bands within each of the plurality of lanes.

43. The computer readable storage media of claim 42 wherein the method further comprises summing the data points along the y-axis.

44. The computer readable storage media of claim 42 wherein the method further comprises normalizing the data points along the x-axis.

45. The computer readable storage media of claim 42 wherein detecting each of the plurality of lanes includes detecting one or more peak values.

46. The computer readable storage media of claim 42 wherein detecting each of a plurality of lanes includes determining a first lateral boundary and a second lateral boundary for each lane.

47. The computer readable storage media of claim 42 wherein detecting one or more bands includes detecting one or more peak values.

48. The computer readable storage media of claim 42 wherein detecting one or more bands includes summing the data points along the x-axis within each of the plurality of lanes.

49. A system comprising:
a first reservoir having a first buffer and a first electrode terminal;
a chamber coupled to the first reservoir and having a bottom plate and a top plate in parallel alignment;
a separation medium disposed between the bottom plate and the top plate;
a second reservoir coupled to the chamber and having a second buffer and a second electrode terminal and having an opening adapted to receive a membrane; and
a sample delivery device having a longitudinal axis and having a plurality of tabs extending orthogonally from the longitudinal axis.

50. The system of claim 49 wherein the sample delivery device includes a membrane.

51. The system of claim 49 wherein the sample delivery device includes a porous membrane.

52. The system of claim 49 wherein the sample delivery device includes a nanoporous membrane.

53. The system of claim 49 wherein the separation medium includes a gel.

54. The system of claim 49 wherein the separation medium includes agarose.

55. The system of claim 49 wherein the separation medium includes agarose having a purity between 0.01 and 30 percent.

56. The system of claim 49 wherein the separation medium includes agarose having a purity between 0.05 and 5.0 percent.

57. The system of claim 49 wherein the separation medium includes linear polyacrylamide (LPA).

58. The system of claim 49 wherein the separation medium includes (LPA) having a purity between 0.05 and 10 percent.

59. The system of claim 49 wherein the separation medium includes a composite of agarose and linear polyacrylamide (LPA).

60. The system of claim 49 wherein the separation medium is adapted to separate molecules in a molecular weight range of between 1,000 to 1,000,000,000.

61. The system of claim 49 wherein the separation medium is adapted to separate molecules in a molecular weight range of between 10,000 to 10,000,000.
